(19) United States
(12) Patent Application Publication
(10) Pub. No.: US 2017/0219507 A1
(43) Pub. Date: Aug. 3, 2017

(54) METHOD OF REAL-TIME DETECTION OF HYDROGEN CONTENT USING OXIDE-BASED HYDROGEN STORAGE ELEMENT HAVING TUNNEL STRUCTURE

(71) Applicant: POSTECH ACADEMY-INDUSTRY FOUNDATION, Pohang-si (KR)

(72) Inventors: Junwoo SON, Pohang-si (KR); Kyuwook IHM, Pohang-si (KR); Hyojin YOON, Siheung-si (KR)

(21) Appl. No.: 15/342,844

(22) Filed: Nov. 3, 2016

(30) Foreign Application Priority Data
Jan. 29, 2016 (KR) 10-2016-0011836

(51) Int. Cl. G01N 27/12; G01N 33/00

(52) U.S. Cl. CPC G01N 27/127; G01N 33/005

(57) ABSTRACT

The present invention provides a method of real-time detection of a hydrogen content using an oxide-based hydrogen storage element having a tunnel structure, wherein the method detects an amount of hydrogen atoms contained in the hydrogen storage element by real-time measuring resistance of the hydrogen storage element including a metal insulator transition (MIT) layer capable of reversibly storing or releasing the hydrogen atoms.

### CROSS-REFERENCE TO RELATED APPLICATION

This application claims the benefit of Korean Patent Application No. 10-2016-0011836, filed on Jan. 29, 2016, in the Korean Intellectual Property Office, the disclosure of which is incorporated herein in its entirety by reference.

### BACKGROUND

Field

The present invention relates to a method of real-time detection of a hydrogen content, and more particularly, to a method of real-time detection of an amount of hydrogen stored in a hydrogen storage element.

### DESCRIPTION OF THE RELATED ART

Currently, a fossil fuel, which has been used as a main fuel, is accompanied by a number of environmental issues, and research into new energy resources has been actively conducted as the depletion of natural resources has emerged. Among the new energy resources, hydrogen is advantageous in that it does not cause pollution during combustion, has a high energy density per weight, and is an infinite resource because hydrogen may be prepared by decomposition of water which accounts for 70% of the Earth's surface.

However, since hydrogen exists as a gas at room temperature and atmospheric pressure, its energy density per volume may not only be low, but storage and transportation may also be dangerous and not easy. Thus, research into the development of a hydrogen storage technique, which may address these limitations, has continued. Also, with respect to a typical hydrogen storage alloy, adsorption and desorption reactions of hydrogen may not only be slow, but also activation energy may be high and hydrogen storage time may be relatively long. In addition, since large volume expansion and contraction occur during adsorption and desorption of hydrogen, defects, such as cracks, may occur when the alloy is used for a long period of time to cause reliability issues.

Furthermore, since there is a risk of explosion when the amount of hydrogen leakage is 4% or more in the atmosphere, there is a need to accurately measure the amount of hydrogen in actual use. Examples of a sensor capable of detecting hydrogen gas may be a ceramic/semiconductor sensor, a semiconductor device type sensor, an optical sensor, and an electrochemical sensor. However, with respect to these hydrogen sensors, it is difficult to detect high-concentration hydrogen gas, their operating temperatures are relatively high, and their performances are easily degraded when used repeatedly. In addition, manufacturing methods may be complicated and difficult.

### SUMMARY

The present invention provides a detection method which may accurately and stably detect hydrogen at a relatively low temperature. However, the problems are exemplary, and the scope of the present invention is not limited by the problems.

According to an aspect of the present invention, there is provided a method of real-time detection of a hydrogen content using an oxide-based hydrogen storage element having a tunnel structure. The method of real-time detection of a hydrogen content using an oxide-based hydrogen storage element having a tunnel structure may detect an amount of hydrogen atoms contained in the hydrogen storage element by real-time measuring resistance of the hydrogen storage element including a metal insulator transition (MIT) layer capable of reversibly storing or releasing the hydrogen atoms.

In the method of real-time detection of a hydrogen content using an oxide-based hydrogen storage element having a tunnel structure, resistance of the MIT layer may be changed while a phase structure of the MIT layer changes from an insulator to a metal or from the metal to the insulator by storing the hydrogen atoms in the MIT layer or releasing the hydrogen atoms to the outside of the MIT layer.

In the method of real-time detection of a hydrogen content using an oxide-based hydrogen storage element having a tunnel structure, the hydrogen storage element may include a metal catalyst formed on the MIT layer, and the metal catalyst may include a plurality of nanoparticles which are spaced apart from each other at a uniform interval and disposed, wherein resistance of the MIT layer may be changed by changing a phase structure of the MIT layer by storing the hydrogen atoms, which are moved to the MIT layer through the nanoparticles, in the MIT layer.

In the method of real-time detection of a hydrogen content using an oxide-based hydrogen storage element having a tunnel structure, the MIT layer may include a vanadium oxide layer, and the vanadium oxide layer may be changed into a vanadium oxyhydride layer by hydrogenation of the vanadium oxide layer so that an amount of the hydrogen atoms stored in the vanadium oxyhydride layer may be increased to increase a resistance value.

In the method of real-time detection of a hydrogen content using an oxide-based hydrogen storage element having a tunnel structure, the hydrogenation may change a phase structure of the MIT layer by storing the hydrogen atoms in the tunnel structure in which vanadium atoms and oxygen atoms of the vanadium oxide layer are missing.

In the method of real-time detection of a hydrogen content using an oxide-based hydrogen storage element having a tunnel structure, the MIT layer may include a vanadium oxyhydride layer, and the vanadium oxyhydride layer may be changed into the vanadium oxide layer by releasing the hydrogen atoms stored in the vanadium oxyhydride layer so that an amount of the hydrogen atoms stored in the vanadium oxyhydride layer may be decreased to decrease a resistance value.

In the method of real-time detection of a hydrogen content using an oxide-based hydrogen storage element having a tunnel structure, a phase structure of the MIT layer may be changed by releasing the hydrogen atoms to the outside of the vanadium oxyhydride layer by annealing the vanadium oxyhydride layer, in which the hydrogen atoms are stored, in an air atmosphere.

According to another aspect of the present invention, there is provided a method of real-time detection of a hydrogen content using an oxide-based hydrogen storage element having a tunnel structure. The method of real-time detection of a hydrogen content using an oxide-based hydrogen storage element having a tunnel structure may include: preparing a hydrogen storage element capable of reversibly storing or releasing hydrogen atoms; storing the hydrogen atoms by hydrogenation by providing a mixed gas containing a hydrogen (H) component to the hydrogen storage element, or releasing the hydrogen atoms stored in the hydrogen storage element to the outside; and detecting an amount of the hydrogen atoms by measuring resistance of the hydrogen storage element in real time during the storing or releasing of the hydrogen atoms in or from the hydrogen storage element.

In the method of real-time detection of a hydrogen content using an oxide-based hydrogen storage element having a tunnel structure, the hydrogen storage element may include a vanadium oxide layer having a rutile structure and a platinum catalyst formed on the vanadium oxide layer, and resistance of the vanadium oxide layer may be changed by changing a phase structure of the vanadium oxide layer by storing an excessive amount of the hydrogen atoms, which are moved to the vanadium oxide layer using a process of lowering an activation barrier through the platinum catalyst, in the vanadium oxide layer.

In the method of real-time detection of a hydrogen content using an oxide-based hydrogen storage element having a tunnel structure, the resistance of the vanadium oxide layer may be changed by changing the phase structure of the vanadium oxide layer, in which the phase structure is changed by releasing the hydrogen atoms stored in the vanadium oxide layer to the outside, to an initial phase structure.

According to an embodiment of the present invention, a method of real-time detection of a hydrogen content using a low-cost oxide-based hydrogen storage element having excellent stability and sensitivity as well as a tunnel structure so as to be able to react rapidly even at a low concentration may be achieved by using a reversible hydrogen storage element which has a simple structure and may be subjected to adsorption and desorption reactions of hydrogen at low temperature. However, the scope of the present invention is not limited by these effects.

### BRIEF DESCRIPTION OF THE DRAWINGS

FIG. 1 schematically illustrates an atomic structure of a metal insulator transition (MIT) layer constituting a hydrogen storage element according to an embodiment of the present invention;

FIG. 2 schematically illustrates a structure of the hydrogen storage element according to the embodiment of the present invention and hydrogen storage and release steps;

FIG. 3 is the result of the analysis of an interface of a hydrogen storage sample according to an experimental example of the present invention using a high-angle annular dark field-scanning transmission electron microscope (HAADF-STEM) and an annular bright-field-scanning transmission electron microscope (ABF-STEM);

FIGS. 4 and 5 are the results of analyzing changes in phase structure of hydrogen storage samples according to the experimental example of the present invention due to hydrogenation;

FIG. 6 is the result of measuring resistance versus time and temperature of the hydrogen storage samples according to the experimental example of the present invention; and

FIG. 7 is the result of analyzing binding energy and photon energy of the hydrogen storage samples according to the experimental example of the present invention in accordance with the presence of hydrogen storage by using X-ray photoelectron spectroscopy (XPS) and near edge X-ray absorption fine structure (NEXAFS) spectroscopy.

### DETAILED DESCRIPTION

Hereinafter, embodiments of the present invention will be described in detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Also, sizes of elements in the drawings may be exaggerated for convenience of explanation.

In the specification, it will be understood that when an element, such as a layer, region, or substrate, is referred to as being “on,” “connected to,” “stacked on” or “coupled to” another element, it can be directly “on,” “connected to,” “stacked on” or “coupled to” the other element or intervening elements may be present. In contrast, when an element is referred to as being “directly on,” “directly connected to” or “directly coupled to” another element, there are no intervening elements present. Like numerals refer to like elements throughout. As used herein, the term “and/or” includes any and all combinations of one or more of the associated listed items.

It will be understood that, although the terms first, second etc. may be used herein to describe various members, components, regions, layers and/or sections, these members, components, regions, layers and/or sections should not be limited by these terms. These terms are only used to distinguish one member, component, region, layer or section from another region, layer or section. Thus, a first member, component, region, layer or section discussed below could be termed a second member, component, region, layer or section without departing from the teachings of the present inventive concept.

Spatially relative terms, such as “above” or “upper” and “below” or “lower”, may be used herein for ease of description to describe one element's relationship to another element(s) as illustrated in the figures. It will be understood that the spatially relative terms are intended to encompass different orientations of the device in use or operation in addition to the orientation depicted in the figures. For example, if the device in the figures is turned over, elements described as “above” other elements would then be oriented “below” the other elements. Thus, the exemplary term “above” can encompass both an orientation of “below” and “above”. The device may be otherwise oriented (rotated 90 degrees or at other orientations) and the spatially relative descriptors used herein interpreted accordingly.

The terminology used herein is for the purpose of describing particular example embodiments only and is not intended to be limiting of the present inventive concept. As used herein, the singular forms are intended to include the plural forms as well, unless the context clearly indicates otherwise. It will be further understood that the terms “comprise” and/or “comprising,” when used in this specification, specify the presence of stated features, integers, steps, operations, members, elements, and/or groups thereof, but do not preclude the presence or addition of one or more other features, integers, steps, operations, members, elements, and/or groups thereof.

Hereinafter, example embodiments are described herein with reference to schematic illustrations of idealized example embodiments. As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing.

A method of real-time detection of a hydrogen content using an oxide-based hydrogen storage element having a tunnel structure according to an embodiment of the present invention may detect an amount of hydrogen atoms contained in the hydrogen storage element by measuring resistance of the hydrogen storage element in real time. The hydrogen storage element includes a metal insulator transition layer (MIT) layer which may reversibly store or release hydrogen atoms. Herein, the MIT layer uses a phase transition material, wherein the MIT layer includes a phase change using a structural phase change between a crystalline phase and an amorphous phase. However, in a case in which the MIT layer is not simply accompanied by the phase change, it will be understood that the MIT layer is a resistance change layer which may be reversibly changed from a high resistance state to a low resistance state.

Since the hydrogen atoms are stored in the MIT layer or the hydrogen atoms are released to the outside of the MIT layer, resistance of the MIT layer is changed while a phase structure of the MIT layer changes from an insulator to a metal or from the metal to the insulator. For example, a resistance value may increase as an amount of the hydrogen atoms stored in the MIT layer increases, or the resistance value may decrease as the amount of the hydrogen atoms stored in the MIT layer decreases.

That is, the method of real-time detection of a hydrogen content may include: preparing a hydrogen storage element capable of reversibly storing or releasing hydrogen atoms, storing the hydrogen atoms by hydrogenation by providing a mixed gas containing a hydrogen (H) component to the hydrogen storage element or releasing the hydrogen atoms stored in the hydrogen storage element to the outside, and detecting an amount of the hydrogen atoms by measuring resistance of the hydrogen storage element in real time during the storing or releasing of the hydrogen atoms in or from the hydrogen storage element. Thus, the method of real-time detection of a hydrogen content, which has excellent stability and sensitivity and may react rapidly even at a low concentration at a low cost, may be achieved by measuring the resistance in real time.

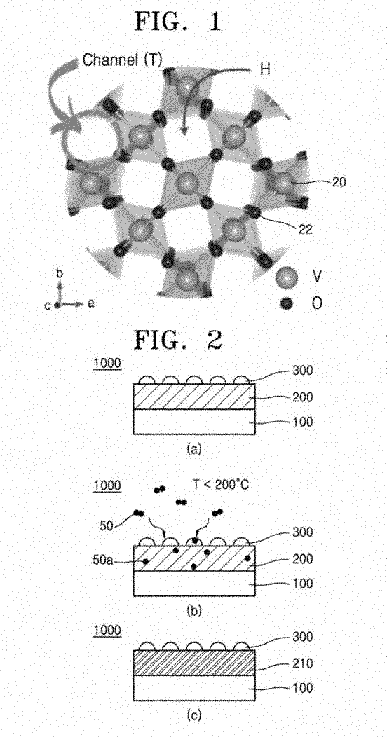

Hereinafter, the hydrogen storage element and the method of real-time detection of a hydrogen content using the same will be described in detail later with reference to FIGS. 1 to 7.

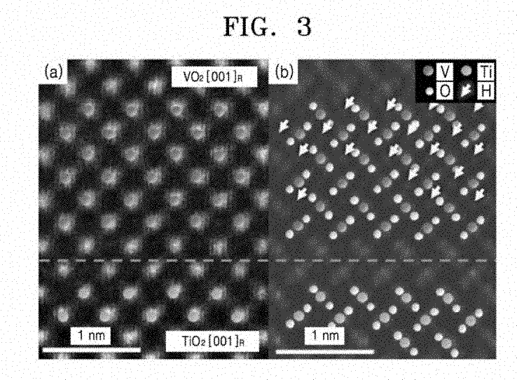

FIG. 1 schematically illustrates an atomic structure of the metal insulator transition (MIT) layer constituting a hydrogen storage element according to an embodiment of the present invention, and FIG. 2 schematically illustrates a structure of the hydrogen storage element according to the embodiment of the present invention and hydrogen storage and release steps.

Referring to FIGS. 1 and 2, an atomic structure of a metal insulator transition (MIT) layer 200 constituting a hydrogen storage element 1000 according to an embodiment of the present invention may be a rutile structure or a distorted rutile structure. The MIT layer 200, for example, may include any one of vanadium oxide (VO₂), niobium oxide (NbO₂), tungsten oxide (WO₂), and titanium oxide (TiO₂).

For example, in a case in which the hydrogen storage element 1000 is formed by using vanadium oxide as the MIT layer 200, the MIT layer 200 includes a structure in which a vanadium atom 20 having a valence of 4 is shared by oxygen atoms 22. The MIT layer 200 includes a channel structure T in which the vanadium atoms 20 and the oxygen atoms 22 are missing along a C-axis ([001] direction). Thus, a hydrogen atom 50a may be easily positioned in the channel structure T.

When the hydrogen atom 50a is positioned in the rutile structure, a vanadium cation is changed from V⁴⁺ to V³⁺ so that the hydrogen atom 50a may be stored, and, since the rutile structure expands slightly when the hydrogen atom 50a is stored, the structure may be modified. Also, a relatively strong hydroxyl bond (OH) may stabilize a hydrogen storage material and the multivalent vanadium cation (V⁴⁺/V³⁺) may facilitate hydrogenation through charge transfer.

Vanadium oxide is one of materials having properties in which it may transform from a metal to an insulator or from the insulator to the metal in a temperature range of about 340K, wherein the vanadium oxide exhibits resistance change characteristics in which resistance is changed by about three orders of magnitude or more near a transition temperature. Thus, the resistance is changed due to changes in phase structure of the vanadium oxide. In a case in which an excessive amount of hydrogen is included, the resistance is changed by five orders of magnitude or more according to the amount of hydrogen, in addition to the temperature.

The structure of the hydrogen storage element 1000 according to the embodiment of the present invention may include a substrate 100, the MIT layer 200, and a metal catalyst 300. Specifically, alumina (Al₂O₃) or titanium oxide (TiO₂), for example, may be used as the substrate 100.

The MIT layer 200, which has a rutile structure and may reversibly store or release hydrogen, may be formed on the substrate 100. The metal catalyst 300 may be formed on the MIT layer 200. The metal catalyst 300 may dissociate a hydrogen molecule 50 into the hydrogen atoms 50a by lowering an activation barrier, and may store the hydrogen atoms 50a in the channel T of the MIT layer 200 by passing the dissociated hydrogen atoms 50a.

The metal catalyst 300 may include a plurality of nanoparticles which are spaced apart from each other at a uniform interval and disposed. Herein, the uniform interval may be an interval in which the dissociated hydrogen atom 50a may move so as to be able to react with the MIT layer 200. Herein, any one of platinum (Pt), palladium (Pd), and gold (Au), for example, may be used as the metal catalyst 300.

In a case in which a single layer of the metal catalyst 300 is formed on the entire surface of the MIT layer 200, since a contact area per volume of the metal catalyst 300 is reduced, storage and release rates of the hydrogen atom 50a may be reduced. Thus, the metal catalyst 300 having a sufficient contact area must be formed so that the hydrogen molecule 50 may rapidly dissociate into the hydrogen atoms 50a by using relatively low energy. In order for the hydrogen atom 50a to be able to move to the MIT layer 200, the metal catalyst 300 may be formed in a nanoparticle size and may be spaced apart from each other at a uniform interval and disposed.

The hydrogen atom 50a may be stored in the MIT layer 200 by hydrogenation of the MIT layer 200 of the hydrogen storage element 1000. For example, the vanadium oxide layer 200 may be formed into a vanadium oxyhydride layer 210 by hydrogenation of the vanadium oxide layer 200.

The hydrogenation may change a phase structure of the vanadium oxide layer 200 by storing the hydrogen atom 50a in the tunnel structure in which the vanadium atoms 20 and the oxygen atoms 22 of the vanadium oxide layer 200 are missing. When the mixed gas (forming gas) including a hydrogen (H) component is provided to the hydrogen storage element 1000, the hydrogen molecule 50 included in the mixing gas is dissociated into the hydrogen atoms 50a by the metal catalyst 300 so that a maximum of one hydrogen atom 50 per two oxygen atoms 22 may be stored in the vanadium oxide layer 200 to be able to maximize energy density per weight and volume stored in the vanadium oxyhydride layer 210.

The hydrogen atoms 50a may be released to the outside of the vanadium oxyhydride layer 210 by annealing the vanadium oxyhydride layer 210 illustrated in (c) of FIG. 2 in the atmosphere and the vanadium oxyhydride layer 210 may be reversibly changed again into the vanadium oxide layer 200 illustrated in (a) of FIG. 2.

That is, changes in volume of the MIT layer 200 occur as the MIT layer 200, as an insulator, is doped with the hydrogen atoms 50a and the resistance is reduced while phase transition of the MIT layer 200, as an insulator, to the MIT layer 200, as a metal, occurs. Thereafter, the resistance increases while the hydrogen atoms 50a stored in the MIT layer 200 react with each other to form the insulating MIT layer 210. Thus, changes in electrical flow occur while the structure of the MIT layer 200 changes according to the storage or release of the hydrogen atoms 50a in the MIT layer 200, and the changes in electrical flow result in changes in the resistance. The reaction not only does not cause defects in the MIT layer 200, but also increases reproducibility in response to hydrogen, and thus, the hydrogen may be reversibly detected in real time.

Hereinafter, an experimental example, to which the above-described technical ideas are applied, will be described to allow for a clearer understanding of the present invention. However, the following experimental example is merely provided to allow for a clearer understanding of the present invention, rather than to limit the scope thereof.

Alumina (Al₂O₃) or titanium oxide (TiO₂) were respectively used as substrates, vanadium oxide (VO₂) was grown on each of the substrates to a thickness of about 30 nm by pulsed laser deposition (PLD), and platinum nanoparticles were deposited by sputtering to prepare hydrogen storage samples.

Thereafter, hydrogen was stored in the vanadium oxide by hydrogenation using a mixed gas (forming gas) containing a hydrogen (H) component, and resistance and structure of the hydrogen storage samples were observed while releasing the hydrogen from the vanadium oxide to the outside by finally annealing at a temperature of 200° C. or less.

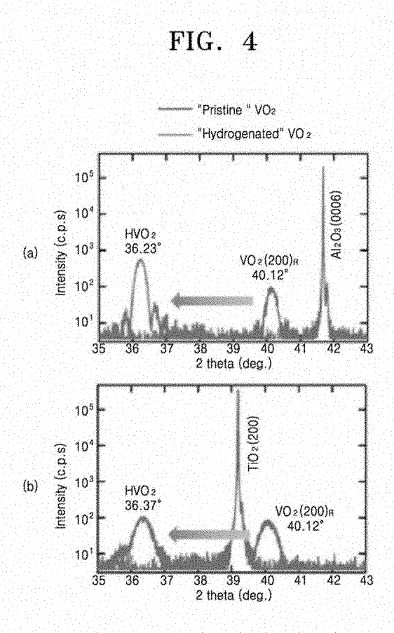

FIG. 3 is the result of the analysis of an interface of the hydrogen storage sample according to an experimental example of the present invention using a high-angle annular dark field-scanning transmission electron microscope (HAADF-STEM) and an annular bright-field-scanning transmission electron microscope (ABF-STEM).

Referring to FIG. 3, the interface of the sample was analyzed according to the presence of hydrogen in the hydrogen storage sample according to the experimental example of the present invention using the HAADF-STEM and ABF-STEM.

(a) of FIG. 3 illustrates a structure of the vanadium oxide (VO₂) formed on the titanium oxide (TiO₂) substrate. (b) of FIG. 3 illustrates that hydrogen atoms filled channels in which vanadium atoms and oxygen atoms were missing. The hydrogen atom may be bonded near to the oxygen atom of the vanadium oxide and may diffuse in a tunnel structure of the vanadium oxide.

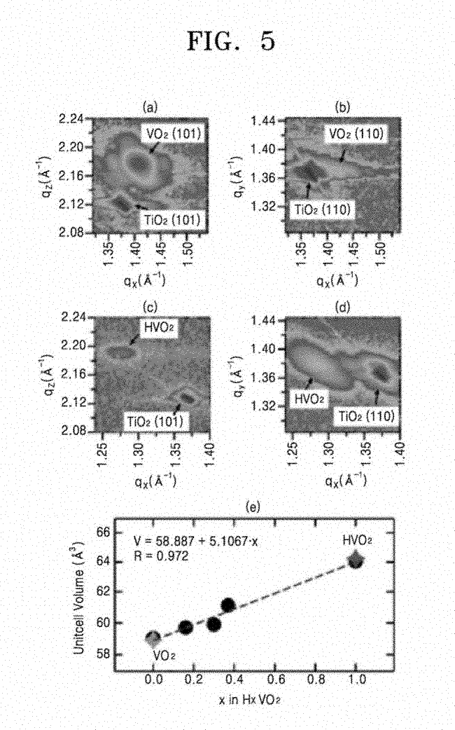

FIGS. 4 and 5 are the results of analyzing changes in phase structure of the hydrogen storage samples according to the experimental example of the present invention due to hydrogenation.

Referring to FIGS. 4 and 5, changes in phase structure of the hydrogen storage samples according to the experimental example of the present invention due to hydrogenation are illustrated. (a) of FIG. 4 is the result of X-ray diffraction analysis of the vanadium oxide formed on the alumina substrate, and (b) of FIG. 4 is the result of X-ray diffraction analysis of the vanadium oxide formed on the titanium oxide substrate.

In all samples, it may be confirmed that a main peak of the vanadium oxide shifted to the left (shift of 2θ value) when a phase change from the vanadium oxide to vanadium oxyhydride occurred after the hydrogenation.

(a) to (d) of FIG. 5 are the result of reciprocal space mapping (RSM) of (101) plane and (110) plane of vanadium oxide according to the presence of hydrogen, and (e) of FIG. 5 is a graph related to a hydrogen concentration per volume of a unit cell.

The vanadium oxide before hydrogen storage was in a state in which it was not stressed due to a large lattice mismatch with the substrate, but, when the vanadium oxide was hydrogenated, the volume of the unit cell expanded about 9.0%. When using this, the concentration of hydrogen stored in the vanadium oxyhydride may be quantified, and, as a result, changes in the volume of the unit cell has a proportional relationship with the hydrogen atom.

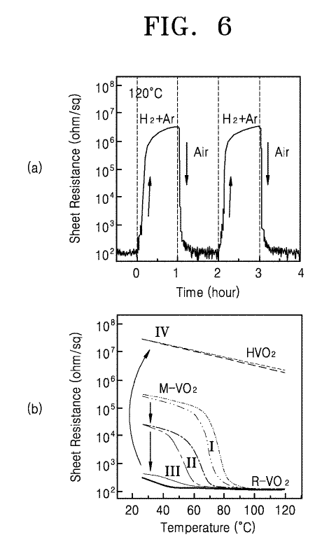

FIG. 6 is the result of measuring resistance versus time and temperature of the hydrogen storage samples according to the experimental example of the present invention.

(a) of FIG. 6 is the result of the in-situ measurement of sheet resistance of the hydrogen storage sample at 120° C., and (b) of FIG. 6 is the result of the in-situ measurement of sheet resistance of vanadium oxyhydride (HₓVO₂) having different hydrogen contents according to the temperature.

Referring to (a) of FIG. 6, vanadium oxide initially had metallic properties with a sheet resistance of 10² Ω/□ and the resistance began to increase while phase transition occurred when hydrogenation began. Thereafter, when a saturation state was reached, the resistance became 10⁷ Ω/□ while a slope began to decrease, and, when hydrogen was released while the vanadium oxide was annealed in the atmosphere, the resistance was decreased. In a case in which hydrogen was reversibly stored and released, it may be understood that hydrogenation and dehydrogenation were repeatedly possible while the hydrogen storage sample showed a resistance difference of five orders of magnitude according to the amount of hydrogen. Thus, it may be confirmed that the vanadium oxide was very stable despite the structural change according to the repeated storage and release of hydrogen.

Referring to (b) of FIG. 6, the resistance was decreased as the temperature was increased, and it may be understood that the resistance was decreased while hydrogen was stored in vanadium oxide as an insulator, and the resistance was rapidly increased when the stored hydrogen and the vanadium oxide reacted with each other to cause a structural change to vanadium oxyhydride (HVO₂) containing an excessive amount of hydrogen.

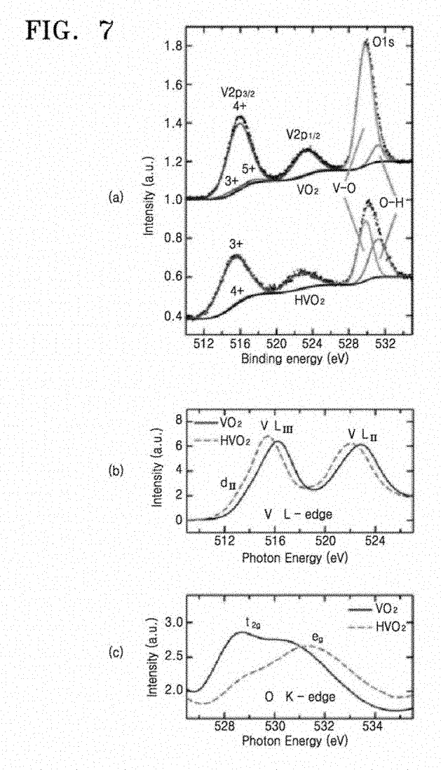

FIG. 7 is the result of analyzing binding energy and photon energy of the hydrogen storage samples according to the experimental example of the present invention in accordance with the presence of hydrogen storage by using X-ray photoelectron spectroscopy (XPS) and near edge X-ray absorption fine structure (NEXAFS) spectroscopy.

Referring to FIG. 7, it may be understood that, with respect to multivalency of vanadium cations, the number of vanadium cations having a valence of 3+ was more than the number of vanadium cations having a valence of 4+ as hydrogen was stored in vanadium oxide and the number of oxygen-hydrogen bonds was also increased. Furthermore, with respect to photon energy of the vanadium cation, it may be understood that a peak shifted from the right to the left as the hydrogenation was performed, and, with respect to photon energy of oxygen anion, it may be understood that a peak shifted from the left to the right as the hydrogenation was performed.

As described above, according to the present invention, a method of accurately detecting a hydrogen content contained in a hydrogen storage element may be simply achieved at a low cost by measuring resistance in real time using the hydrogen storage element including a vanadium oxide layer capable of reversibly storing or releasing hydrogen atoms and platinum (Pt) nanoparticles which are formed on the vanadium oxide layer to be able to dissociate a hydrogen molecule into hydrogen atoms.

Although the present invention has been described with reference to the embodiment illustrated in the accompanying drawings, it is merely illustrative, and those skilled in the art will understand that various modifications and equivalent other embodiments of the present invention are possible. Thus, the true technical protective scope of the present invention should be determined by the technical spirit of the appended claims.

1. A method of real-time detection of a hydrogen content using an oxide-based hydrogen storage element having a tunnel structure, wherein the method detects an amount of hydrogen atoms contained in the hydrogen storage element by real-time measuring resistance of the hydrogen storage element including a metal insulator transition (MIT) layer capable of reversibly storing or releasing the hydrogen atoms.

2. The method of claim 1, wherein resistance of the MIT layer is changed while a phase structure of the MIT layer changes from an insulator to a metal or from the metal to the insulator by storing the hydrogen atoms in the MIT layer or releasing the hydrogen atoms to outside of the MIT layer.

3. The method of claim 1, wherein the hydrogen storage element comprises a metal catalyst formed on the MIT layer, and the metal catalyst comprises a plurality of nanoparticles which are spaced apart from each other at a uniform interval and disposed,
wherein resistance of the MIT layer is changed by changing a phase structure of the MIT layer by storing the hydrogen atoms, which are moved to the MIT layer through the nanoparticles, in the MIT layer.

4. The method of claim 1, wherein the MIT layer comprises a vanadium oxide layer, and
the vanadium oxide layer is changed into a vanadium oxyhydride layer by hydrogenation of the vanadium oxide layer so that an amount of the hydrogen atoms stored in the vanadium oxyhydride layer is increased to increase a resistance value.

5. The method of claim 4, wherein the hydrogenation changes a phase structure of the MIT layer by storing the hydrogen atoms in the tunnel structure in which vanadium atoms and oxygen atoms of the vanadium oxide layer are missing.

6. The method of claim 1, wherein the MIT layer comprises a vanadium oxyhydride layer, and
the vanadium oxyhydride layer is changed into the vanadium oxide layer by releasing the hydrogen atoms stored in the vanadium oxyhydride layer so that an amount of the hydrogen atoms stored in the vanadium oxyhydride layer is decreased to decrease a resistance value.

7. The method of claim 6, wherein a phase structure of the MIT layer is changed by releasing the hydrogen atoms to outside of the vanadium oxyhydride layer by annealing the vanadium oxyhydride layer, in which the hydrogen atoms are stored, in an air atmosphere.

8. A method of real-time detection of a hydrogen content using an oxide-based hydrogen storage element having a tunnel structure, the method comprising:
preparing a hydrogen storage element capable of reversibly storing or releasing hydrogen atoms;
storing the hydrogen atoms by hydrogenation by providing a mixed gas containing a hydrogen (H) component to the hydrogen storage element, or releasing the hydrogen atoms stored in the hydrogen storage element to outside; and
detecting an amount of the hydrogen atoms by measuring resistance of the hydrogen storage element in real time during the storing or releasing of the hydrogen atoms in or from the hydrogen storage element.

9. The method of claim 8, wherein the hydrogen storage element comprises a vanadium oxide layer having a rutile structure and a platinum catalyst formed on the vanadium oxide layer, and
resistance of the vanadium oxide layer is changed by changing a phase structure of the vanadium oxide layer by storing the hydrogen atoms, which are moved to the vanadium oxide layer using a process of lowering an activation barrier through the platinum catalyst, in the vanadium oxide layer.

10. The method of claim 9, wherein the resistance of the vanadium oxide layer is changed by changing the phase structure of the vanadium oxide layer, in which the phase structure is changed by releasing the hydrogen atoms stored in the vanadium oxide layer to the outside, to an initial phase structure.
